(19) United States
(12) Patent Application Publication
(10) Pub. No.: US 2007/0165873 A1
(43) Pub. Date: Jul. 19, 2007

(54) Communications unit comprising a gravitation switch

(76) Inventor: Ole Birch, Naerum (DK)

Correspondence Address: ALTERA LAW GROUP, LLC, 6500 CITY WEST PARKWAY, SUITE 100, MINNEAPOLIS, MN, 55344-7704, US

(21) Appl. No.: 10/572,902

(22) PCT Filed: Sep. 15, 2004
(86) PCT No.: PCT/DK04/00610

§ 371 Date: Sep. 6, 2006

(30) Foreign Application Priority Data
Sep. 26, 2003 (DK) PA 2003 01400

(51) Int. Cl. A61F 11/06

(52) U.S. Cl. 381/71.6

(57) ABSTRACT

A communications unit, preferably a headset, has a number of control knobs (2, 3) for adjusting the electrical properties of the headset and is suspended by an ear hook, which may be adjusted such that the headset may be used on both the left and the right ear, said headset when switching from one ear to the other being rotated 180°, while the position of the ear hook is unchanged. In order to ensure that the functions of the control knobs (2, 3) relative to their positions on the headset are the same, no matter whether the headset is carried on the left or the right ear, a gravitation switch is arranged in a printed circuit board which is mounted in the headset. The gravitation switch of the headset provides for automatic switching of the functions of the control knobs without intervention by the user, when the headset is switched from a position on one ear to the other ear.

The invention relates to a communications unit having an electrical circuit comprising a printed circuit board, wherein the communications unit has a number of control knobs for adjusting the electrical properties of the communications unit, and wherein the functions of the control knobs may be adapted in dependence on the orientation of the communications unit.

Communications units, such as headsets for use in connection with telephony, be it mobile telephony or public switched telephony, from being passive units, are today equipped with electronics and control knobs, so that various functions, such as volume control, answering of calls, interruption of calls, etc., may be initiated directly from the headset.

The headsets are moreover of compact structure and are made inter alia in lightweight versions which may be placed on the left or the right ear by means of an ear hook.

It is noted in this connection that a headset with an ear hook to be used on the right as well as the left ear requires that the headset itself is rotated 180°, which means that control knobs offset relative to the horizontal axis of symmetry of the headset will change their positions.

This may be a discomfort to users if e.g. a knob for volume control, which is normally disposed lowermost, changes its position to being disposed uppermost.

A solution to this problem is found in WO 01/37524, where logic circuits, which may optionally be controlled from a mobile telephone, are adapted to configure the control knobs for use in connection with switching the headset from one ear to the other.

Accordingly, an object of the invention is to provide a simple change of the functions of the control knobs of the communications unit, so that if the position of the communications unit and thereby of the control knobs is changed, then the functions of the control knobs will be changed mutually such that a user does not notice a change in the functions of the control knobs relative to their positions.

The object of the invention is achieved by a communications unit of the type defined in the introductory portion of claim 1, which is characterized in that the printed circuit board has incorporated therein a gravitation switch which is adapted to switch the functions of the control knobs. Hereby, the changes in the functions of the control knobs take place without intervention by a user.

A particularly expedient communications unit in the implementation of the invention is achieved if, as stated in claim 2, it is formed by a headset which may be placed on the right or the left ear.

It is additionally expedient, as stated in claim 3, that the gravitation switch comprises an elongated channel or a set of channels in the printed circuit board, that a movable conducting object is arranged in the channel or the set of channels, and that through-platings are provided at the ends of the channel or the set of channels, so that the movable conducting object when contacting the through-platings contacts the connection between the through-platings and interrupts the connection when it leaves the through-platings.

Hereby, a number of automatic switching functions of the control knobs of the headset may be provided merely by orienting the headset in a given direction.

When, as stated in claim 4, the channel is oriented vertically, a simple structure of a switching arrangement is achieved, when a headset is switched for use from the left ear to the right ear, or vice versa.

To improve the flexibility of the communications unit additionally, it is an advantage if, as stated in claim 5, the set of channels is configured as three sub-channels in a star configuration, which provides for more automatic settings of the functionalities of the headset.

When, as stated in claim 6, the conducting object is formed by a ball or a cylinder of conducting rubber, a steady and noiseless switching of the functions of the headset is achieved.

When switching the functions of the control knobs of the headset when the headset is placed on the one ear and is switched to the other ear, the changes in the functions of the control knobs are performed if, as stated in claim 7, the number of control knobs is two and the gravitation switch comprises the channel with the conducting object which, when the conducting object is at one end of the channel, controls a switching circuit which will cause the uppermost control knob to perform a first function and the lowermost one to perform a second function, and when the gravitation switch is at the opposite end of the housing, corresponding to the uppermost control knob switching to being the lowermost control knob and the lowermost control knob to being the uppermost control knob, then the switching circuit will cause the uppermost and lowermost control knobs to still perform the first function and the second function, respectively.

Finally, it is an advantage if, as stated in claim 8, two of the channels in the set of channels are arranged symmetrically relative to the horizontal and extend obliquely to the same side relative to the vertical, while the third channel extends horizontally, as the third channel may then be used as a connection to a charging circuit when the third channel of the headset is disposed vertically.

The invention will then be explained more fully with reference to the drawing, in which

FIG. 1 shows the principle in the switching of the control knobs of a communications unit,

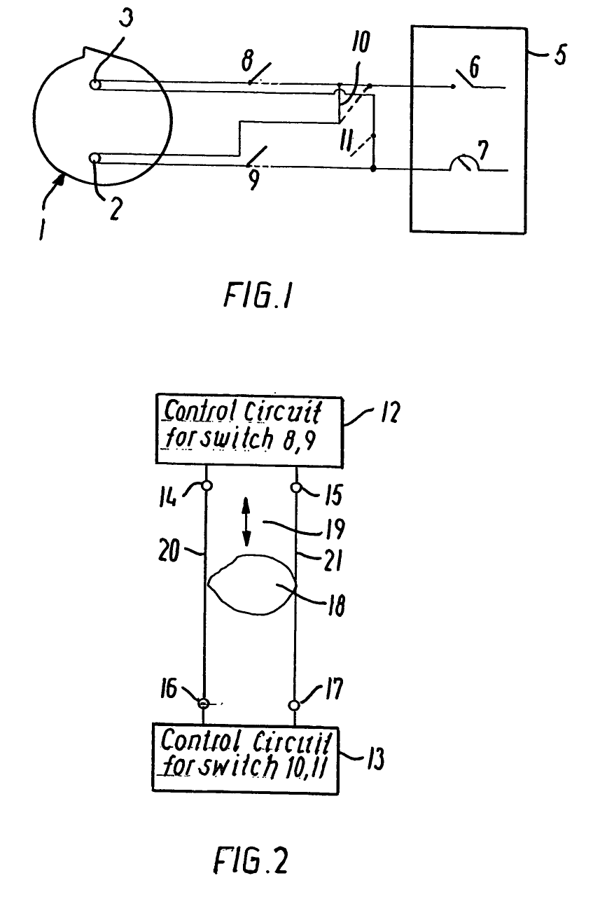

FIG. 2 shows the principle in a first embodiment in the configuration of a gravitation switch with a channel for a headset according to the invention, while

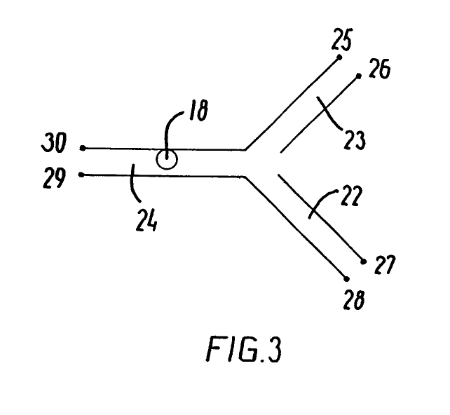

FIG. 3 shows a set of channels for use in a gravitation switch according to the invention.

In FIG. 1, the numeral 1 designates a headset, e.g. of the type known from WO 01/86923, which may be used by a user on the right as well as the left ear, as the ear hook of the headset may be mounted in two positions, there being a difference of 180° between the positions. When switching from one ear to the other, the headset itself must also be rotated 180°, as the loudspeaker of the headset must be directed toward the user's ear.

As will be seen in FIG. 1, the headset has control knobs 2, 3. Further, a circuit 5 is shown schematically, which is shown outside the headset for clarity, but is incorporated in it in practice. This circuit inter alia has an on/off switch 6 and a volume control 7, which may be operated by means of the control knobs 2 and 3.

The circuit 5 is built on a printed circuit board, and this printed circuit board has embedded therein a gravitation switch, whose basic structure is shown in FIG. 2.

The gravitation switch consists of a conducting object, such as a ball 18 or a cylinder of conducting rubber 18, which may be moved inside a channel 19, defined by two walls 20, 21, between a first set of through-platings 14, 15 on the printed circuit board and a second set of through-platings 16, 17 on the printed circuit board. The gravitation switch is embedded in the printed circuit board such that it is parallel or almost parallel with the control knobs 2, 3, cf. FIG. 1. Control circuits 12, 13 are connected to the two sets of through-platings 14, 15 and 16, 17.

These control circuits are adapted to control the four switches, which are designated 8, 9, 10, 11 in FIG. 1, in the following manner.

If the conducting ball 18 short-circuits the through-platings 14, 15, then the switches 8, 9 will be open, While the switches 10, 11 will be closed, which means that the volume control 7 may be operated by the control knob 3, while the on/off switch 6 may be operated by the control knob 2.

If the headset is turned when switching from one ear to the other, then the ball 18 will leave the through-platings 14,1 5 and instead short-circuit the through-platings 16, 17, which then causes the switches 8, 9 to close and the switches 10, 11 to open, which means that the control knob 3 activates the on/off switch 6, while the control knob 2 activates the volume control 7.

The functions of the control knobs relative to their positions may thus be maintained, no matter whether the headset is rotated physically.

It is noted that the control circuits shown in FIG. 2 may be implemented as a single microprocessor which is informed of the orientation of the headset via the through-platings and, on the basis of this, may implement the switching of the control knobs from controlling one functionality to another functionality.

FIG. 3 shows a set of channels which provide more functions than the channel in FIG. 2.

This figure also shows the conducting ball 18 which may be moved in a set of channels consisting of three channels 22, 23, and 24.

Each of the channels has through-platings which are designated 27, 28 for the channel 22, are designated 25, 26 for the channel 23, while the through-platings of the channel 24 are designated 29, 30.

In this configuration, the channels 22, 23 may expediently be used for switching the functions of the control knobs in dependence on whether a headset is used on the right or the left ear, while the last channel 24 may e.g. be used for connection of a charging circuit to the headset, if it is e.g. arranged in a holder so that the channel 24 is oriented vertically.

Although the invention has been explained in connection with two control knobs, nothing within the scope of the claims prevents it from being implemented with more control knobs, as the control circuits may readily be dimensioned for controlling the functions of several switches.

Also, more channels than three may be introduced into the set of channels.

1. A communications unit having an electrical circuit (5) comprising a printed circuit board, wherein the communications unit has a number of control knobs (2, 3) for adjusting the electrical properties of the communications unit, and wherein the functions of the control knobs (2, 3) may be adapted in dependence on the orientation of the communications unit, characterized in that the printed circuit board has incorporated therein a gravitation switch (18, 19) which is adapted to switch the functions of the control knobs (2, 3).

2. A communications unit according to claim 1, characterized in that it is formed by a headset which may be placed on the right or the left ear.

3. A communications unit according to claim 1 or 2, characterized in that the gravitation switch (18, 19) comprises an elongated channel (19) or a set of channels (22, 23, 24) in the printed circuit board, that a movable conducting object (18) is arranged in the channel or set of channels, and that through-platings (14, 15, 16, 17) are provided at the ends of the channel or the set of channels, so that the movable conducting object (18) when contacting the through-platings contacts the connection between the through-platings and interrupts the connection when it leaves the through-platings.

4. A communications unit according to claim 3, characterized in that the channel (19) is oriented vertically.

5. A communications unit according to claim 3, characterized in that the set of channels (22, 23, 24) is configured as three sub-channels in a star configuration.

6. A communications unit according to claims 1-5, characterized in that the conducting object (18) is formed by a ball or a cylinder of conducting rubber.

7. A communications unit according to claims 3-6, characterized in that the number of control knobs (2, 3) is two, and that the gravitation switch (18, 19) comprises the channel (19) with the conducting object (18) which, when the conducting object is at one end of the channel, controls a switching circuit (12, 13) which will cause the uppermost control knob (3) to perform a first function and the lowermost one (2) to perform a second function, and when the gravitation switch is at the opposite end of the housing, corresponding to the uppermost control knob (3) switching to being the lowermost control knob (2) and the lowermost control knob to being the uppermost control knob, then the switching circuit will cause the uppermost and lowermost control knobs to still perform the first function and the second function, respectively.

8. A communications unit according to claim 5, characterized in that two of the channels (22, 23) in the set of channels are arranged symmetrically relative to the horizontal and extend obliquely relative to the vertical, while the third channel (24) extends horizontally.
